(19) United States
(12) Patent Application Publication
(10) Pub. No.: US 2026/0274860 A1
(43) Pub. Date: Sep. 17, 2026

(54) PROCESS FOR MANUFACTURE OF A CYCLOPENTA THIOPHENE DERIVATIVE

(71) Applicant: Boehringer Ingelheim International GmbH, Ingelheim am Rhein (DE)

(72) Inventors: Christoph Paul WETZEL, Ehingen (DE); Bodo BETZEMEIER, Biberach an der Riss (DE); Pol KARIER, Oberaach (CH); Thomas KRUEGER, Kisslegg (DE)

(21) Appl. No.: 19/560,221

(22) Filed: Mar. 9, 2026

(30) Foreign Application Priority Data
Mar. 12, 2025 (EP) 25163331.9

(51) Int. Cl. C07D 495/04; C07D 333/78

(52) U.S. Cl. CPC C07D 495/04; C07D 333/78

(57) ABSTRACT

The present invention relates to a novel process for preparing a compound of formula 4:

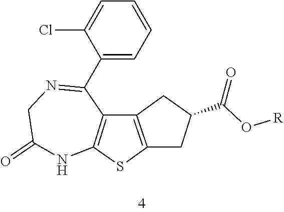

wherein R is defined herein. The present invention also relates to intermediates useful for preparing 4.

### FIELD OF THE INVENTION

The present invention relates to a process for the manufacture of a specific cyclopentathiophene derivative, namely alkyl (4S)-13-(2-chlorophenyl)-10-oxo-7-thia-9,12-diazatricyclo[6.5.0.0^{2,6}]trideca-1(8),2(6),12-triene-4-carboxylate (4), which is a key intermediate for preparing the compounds described in WO 2022/101377. The invention also relates to intermediates of said manufacturing process.

### BACKGROUND OF THE INVENTION

Synthesis of compound 4a was previously described in WO 2022/10377 and DE 3909012 (Scheme 1). The main drawback of the said synthesis process is the use of bromoacetyl bromide, which is corrosive with a potential danger of explosion (Bromoacetyl bromide; SDS No. 1907/2006 [Online]; Sigma-Aldrich; March 2024). Furthermore, bromoacetyl bromide is a suspected genotoxin with its hydrolysis product bromoacetic acid having been reported to impair cytoskeleton architecture and cause epigenetic modification (Chem. Biol. Interact. 2022, 368, 110192) and also to damage double stranded DNA in human cells (Environ. Mol. Mutagen. 2010, 51, 205). Additionally, cyclization of compound 3a to compound 4a requires the reaction mixture being stirred at reflux for 24 hours in the presence of silica gel, which causes abrasion to the reaction vessel when performed at technical scale.

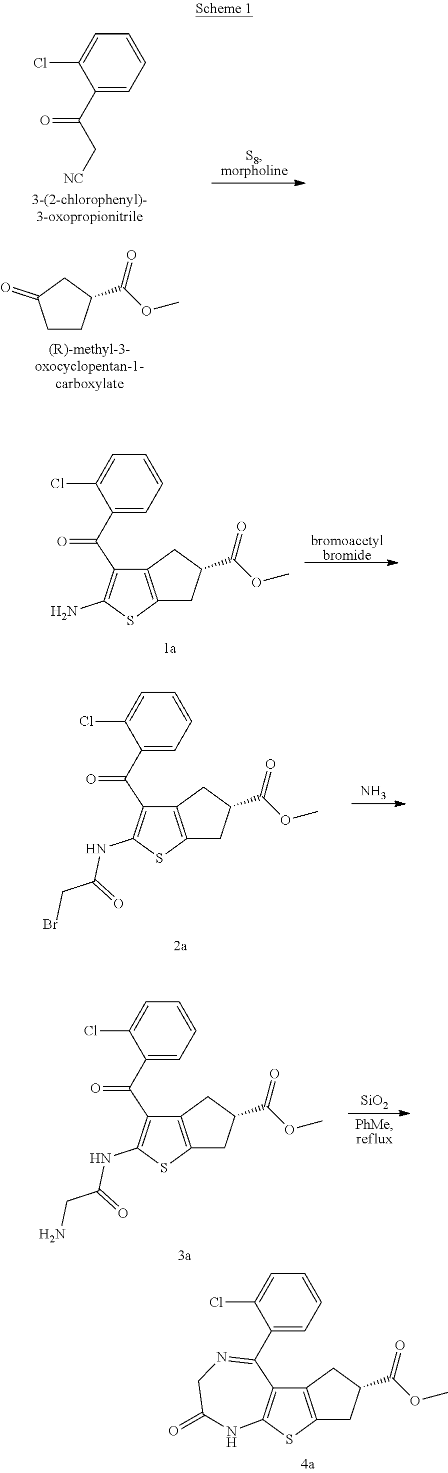

### DETAILED DESCRIPTION

The present invention provides a process for manufacture of compound 4

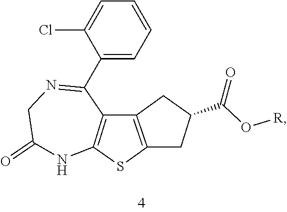

wherein R is C₁₋₃-alkyl,
comprising the following reaction steps:
   (a) reacting compound 1, wherein R is C₁₋₃-alkyl, and N-(tert-butyloxycarbonyl)-glycine (N-Boc-glycine) in the presence of a suitable amide coupling reagent, preferably 1,1′-carbonyldiimidazole, in a suitable solvent to obtain compound 2, wherein R is C₁₋₃-alkyl and Boc is tert-butyloxycarbonyl;

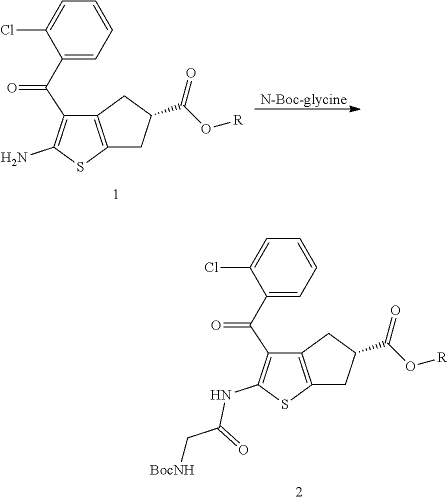

(b) optionally isolating compound 2;
(c) reacting compound 2, wherein R is C₁₋₃-alkyl and Boc is tert-butyloxycarbonyl, with a suitable acid, preferably methanesulfonic acid, in a suitable solvent to obtain compound 3, wherein R is C₁₋₃-alkyl;

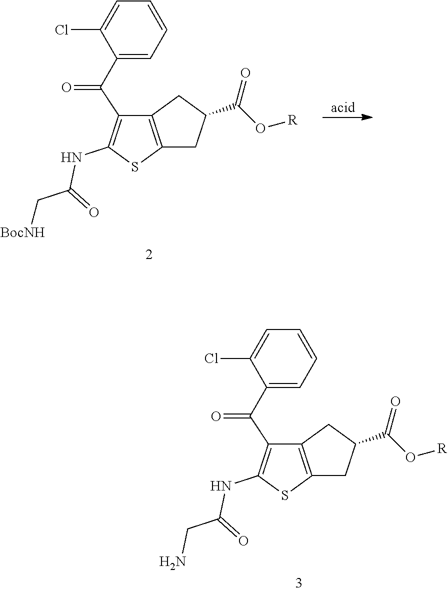

(d) optionally isolating compound 3;
(e) reacting compound 3, wherein R is C₁₋₃-alkyl, with a suitable acid, preferably pivalic acid, in a suitable solvent to obtain compound 4, wherein R is C₁₋₃-alkyl;

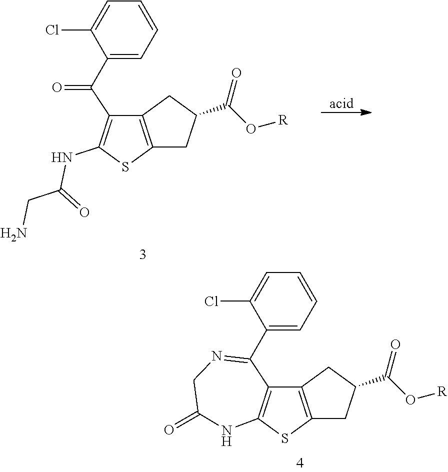

and
   (f) isolating compound 4.

Another aspect of the present invention relates to the above process further comprising the following reaction steps to be carried out prior to step (a):
(g) reacting 3-(2-chlorophenyl)-3-oxo-propionitrile and (R)-alkyl-3-oxocyclopentan-1-carboxylate, wherein R is C₁₋₃-alkyl, in the presence of sulfur and a suitable base, preferably morpholine, in a suitable solvent to obtain compound 1, wherein R is C₁₋₃-alkyl;

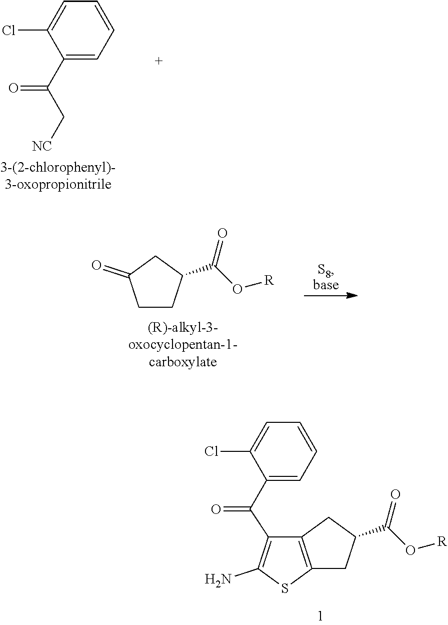

(h) optionally isolating compound 1.

In the above process, (R)-methyl-3-oxocyclopentan-1-carboxylate is preferably used for step (g). Correspondingly, in Scheme 2 below, as well as in reaction steps (a) to (h) above, R is C₁₋₃-alkyl, preferably methyl.

The process according to the invention can be used in technical scale.

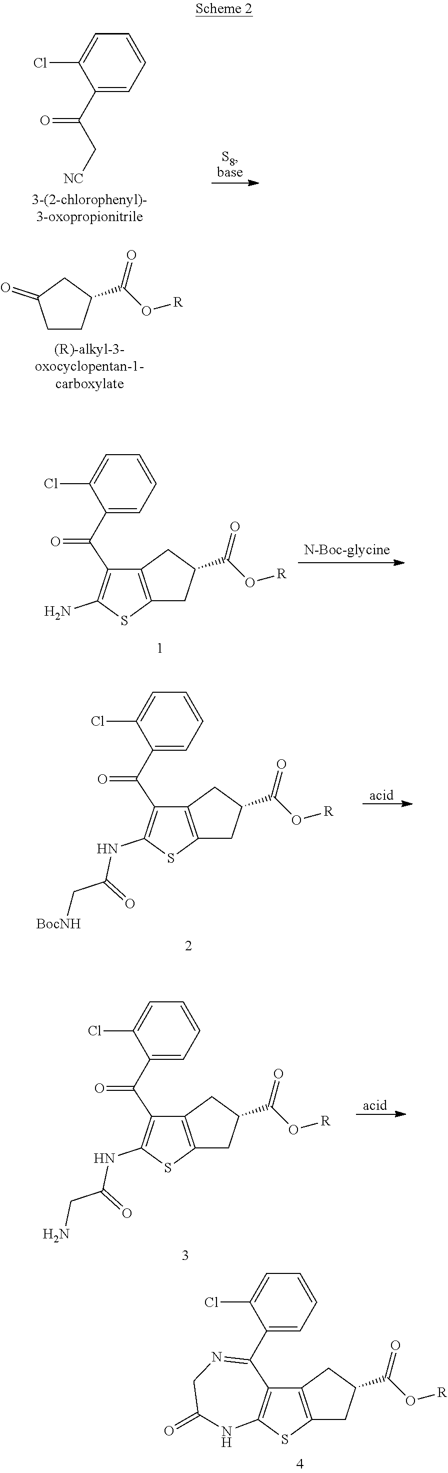

The process according to the present invention has the following advantages over the process of the prior art:
1) avoidance of hazardous reagents, particularly of bromoacetyl bromide
2) all steps can be easily carried out on technical scale
3) preparation of compound 4 with high purity in high overall yield
4) avoidance of cumbersome chromatographic purification

Suitable amide coupling reagent for step (a) include 1,1′-carbonyldiimidazole, propanephosphonic acid anhydride, and 1-ethyl-3-(3-dimethylaminopropyl)-carbodiimide. Preferably, 1,1′-carbonyldiimidazole is used.

Suitable solvents for step (a) include toluene, isopropyl acetate, N,N-dimethylformamide, and mixtures thereof. Preferably, toluene is used.

Step (a) is preferably carried out at a temperature between 50° C. and 110° C., more preferably between 70° C. and 90° C.

Step (a) is carried out with 1,1′-carbonyldiimidazole as the amide coupling reagent and toluene as the solvent at a temperature preferably between 50° C. and 110° C., more preferably between 70° C. and 90° C.

Isolation step (b) can be carried out with water as the antisolvent, followed by isolation via filtration or centrifugation.

Suitable acids for step (c) include methanesulfonic acid, 4-toluenesulfonic acid, and trifluoromethanesulfonic acid. Preferably, methanesulfonic acid is used.

Suitable solvents for step (c) include toluene, acetonitrile, methanol, xylene, and mixtures thereof. Preferably, a mixture of toluene and acetonitrile is used.

Step (c) is preferably carried out at a temperature between 20° C. and 100° C., more preferably between 50° C. and 70° C.

Step (c) is carried out with methanesulfonic acid as the acid and a mixture of toluene and acetonitrile as the solvent at a temperature preferably between 20° C. and 100° C., more preferably between 50° C. and 70° C.

Suitable acids for step (e) include pivalic acid, benzoic acid, and 4-methylbenzoic acid. Preferably, pivalic acid (2,2,-dimethylpropanoic acid) is used.

Suitable solvents for step (e) include 2-methylbutan-2-ol, methanol, acetonitrile, toluene, and mixtures thereof. Preferably, 2-methylbutan-2-ol is used.

Step (e) is preferably carried out at a temperature between 60° C. and 110° C., more preferably between 75° C. and 95° C.

Step (e) is carried out with pivalic acid is used as the acid and 2-methylbutan-2-ol as the solvent at a temperature preferably between 60° C. and 110° C., more preferably between 75° C. and 95° C.

Step (f) is preferably carried out by aqueous work up and subsequent recrystallization in acetonitrile.

Suitable base for step (g) include amine bases such as morpholine, diisopropylethylamine, and N-methyl morpholine. Preferably, morpholine is used.

Suitable solvents for step (g) include N,N-dimethylformamide, methanol, 1,3-dimethyl-2-imidazolidinone, and mixtures thereof. Preferably, N,N-dimethylformamide is used.

Step (g) is preferably carried out at a temperature between 20° C. and 120° C., more preferably at a temperature between 40° C. and 70° C.

Step (g) is carried out with morpholine as the base and N,N-dimethylformamide as the solvent at a temperature preferably between 20° C. and 120° C., more preferably between 40° C. and 70° C.

### General Definitions

Terms not specifically defined herein should be given the meanings that would be given to them by one of skill in the art in light of the disclosure and the context.

In case a compound of the present invention is depicted in the form of a chemical name and as a formula, the formula shall prevail in case of any discrepancy.

Throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates. The process described herein is also applicable to the corresponding starting materials, intermediates, and final products of the opposite absolute configuration, and for mixtures of the enantiomers.

### Alkyl

The term “C₁₋₃-alkyl”, either alone or in combination with another radical, denotes an acyclic, saturated, branched or linear hydrocarbon radical with 1 to 3 C atoms. The term C₁₋₃-alkyl embraces the radicals H₃C—, H₃C—CH₂—, H₃C—CH₂—CH₂—, and H₃C—CH(CH₃)—.

### EXPERIMENTAL

Abbreviations:

| | |
|---|---|
| ° C. | degree Celsius |
| g | gram(s) |
| kg | kilogram(s) |
| L | liter |
| Me | methyl |

Abbreviations within analytical data:

| | |
|---|---|
| ¹H-NMR | proton nuclear magnetic resonance |
| δ | chemical shift |
| DMSO-d₆ | hexa-deutero-dimethylsulfoxide |
| MHz | megahertz |
| ppm | parts per million |
| s | singlet |
| d | doublet |
| t | triplet |
| m | multiplet |
| ESI | electrospray ionization |
| APCI | atmospheric pressure chemical ionization |

Substances used for seeding in Examples hereinbelow can be prepared according to the process described in WO 2022/10377.

### Example 1

### Synthesis of methyl (5S)-2-amino-3-(2-chlorobenzoyl)-4H,5H,6H-cyclopenta[b]thiophene-5-carboxylate (1a)

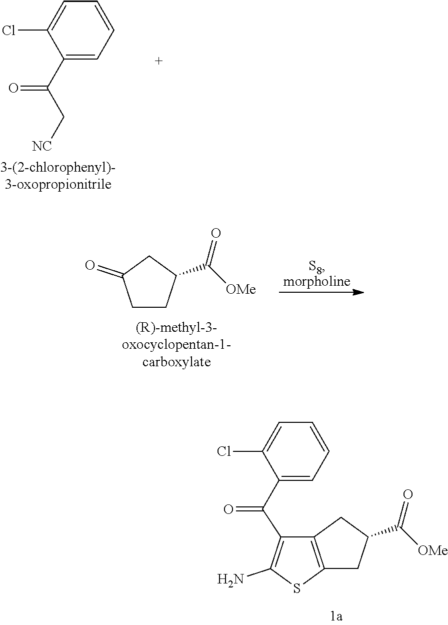 [SMILES: N#CCC(=O)c1ccccc1Cl ; COC(=O)[C@@H]1CCC(=O)C1 ; "S8,morpholine" ; COC(=O)[C@@H]1Cc2sc(N)c(C(=O)c3ccccc3Cl)c2C1]

At 20° C., 50.00 kg (278.4 mol) 3-(2-chlorophenyl)-3-oxo-propionitrile and 8.93 kg (278.4 mol) sulfur are suspended in 200 L N,N-dimethylformamide. A solution of 41.55 kg (292.3 mol) (R)-methyl-3-oxocyclopentan-1-carboxylate in 15 L N,N-dimethylformamide is added. Additional 35 L of N,N-dimethylformamide are added. The reaction mixture is heated to 50° C. before a solution of 12.12 kg (139.2 mol) morpholine in 15 L N,N-dimethylformamide is added at this temperature. Additional 35 L of N,N-dimethylformamide are added at 50° C. before the mixture is heated to 60° C. The reaction mixture is stirred for 1 hour at 60° C. 125 L of water are added at 60° C. The mixture is seeded with 50 g of compound 1a before additional 100 L water is added. Finally, within a period of approximately 2 hours, the reaction mixture is cooled to 20° C. and stirred for an additional period of 15 minutes at this temperature. The product is collected via centrifugation, washed with 150 L of methanol and dried in a vacuum oven at 50° C.

Yield: 40.99 kg (58% of theory) of compound 1a as a solid.

1H-NMR (400 MHz, DMSO-d₆) δ (ppm)=8.57 (s, 2H), 7.39-7.52 (m, 3H), 7.29-7.31 (m, 1H), 3.55 (s, 3H), 3.36-3.45 (m, 1H), 2.73-2.93 (m, 2H), 1.90-2.06 (m, 2H). MS (ESI⁺/APCI⁺): 336 (M+H)⁺.

### Example 2

### Synthesis of methyl (5S)-2-(2-{[(tert-butyloxy)carbonyl]amino}acetamido)-3-(2-chlorobenzoyl)-4H,5H,6H-cyclopenta[b]thiophene-5-carboxylate (2b)

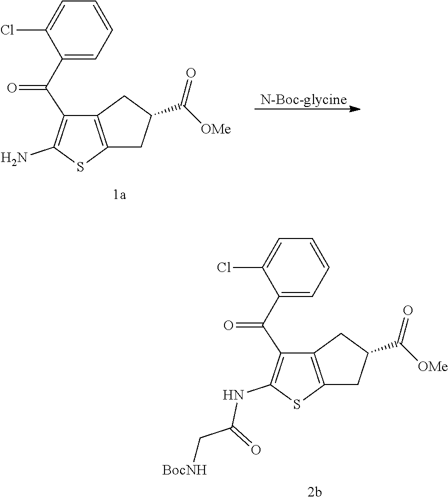 [SMILES: COC(=O)[C@@H]1Cc2sc(N)c(C(=O)c3ccccc3Cl)c2C1 ; "N-Boc-glycine" ; COC(=O)[C@@H]1Cc2sc(NC(=O)CNC(=O)OC(C)(C)C)c(C(=O)c3ccccc3Cl)c2C1]

At 20° C., 43.46 kg (268.0 mol) of 1,1-carbonyldiimidazole are added in four portions to a stirred suspension of 46.69 kg (268.0 mol) N-(tert-butyloxycarbonyl)glycine (N-Boc-glycine) in 150 L toluene in a first reactor, and the resulting solution is stirred for 30 minutes at 20° C. 50.00 kg (148.9 mol) of compound 1a is suspended in 175 L toluene in a second reactor. The mixture in the second reactor is heated to 80° C. before the solution from the first reactor is added at this temperature. The first reactor is rinsed with 25 L of toluene and further added. The solution is stirred for 2 hours at 80° C. After cooling to 20° C., a solution of 15.6 kg citric acid monohydrate in 140 L water is added.

The phases are separated, and the aqueous phase is discarded. An additional solution 15.6 kg citric acid monohydrate in 140 L water is added. The phases are again separated, and the aqueous phase is discarded. 100 L of water are added, and the phases are separated. The aqueous phase is discarded. 200 L solvent of the resulting organic phase is distilled off under reduced pressure, and 225 L of methylcyclohexane and 12.5 L of methanol are added at 50° C. The solution is seeded with 184 g of compound 2b. The mixture is cooled to 0° C. and stirred for 1 hour at this temperature. The product is collected via centrifugation, washed with a mixture of 75 L toluene and 75 L methylcyclohexane and dried in a vacuum oven at 50° C.

Yield: 71.42 kg (79% of theory) of compound 2b as a solid.

¹H-NMR (400 MHz, DMSO-d₆) δ (ppm)=12.25 (s, 1H), 7.74 (t, 1H), 7.48-7.61 (m, 3H), 7.40-7.42 (m, 1H), 3.88 (d, 2H), 3.56 (s, 3H), 3.49-3.54 (m, 1H), 2.95-3.13 (m, 2H), 2.03-2.21 (m, 2H), 1.31-1.41 (m, 9H).

MS (ESI⁺/APCI⁺): 493 (M+H)⁺.

### Example 3

### Synthesis of methyl (4S)-13-(2-chlorophenyl)-10-oxo-7-thia-9,12-diazatricyclo[6.5.0.0^{2,6}]trideca-1(8),2(6),12-triene-4-carboxylate (4a)

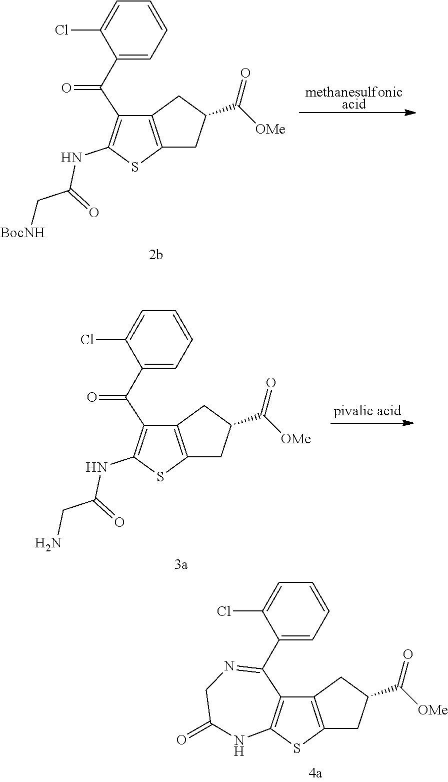 [SMILES: COC(=O)[C@@H]1Cc2sc(NC(=O)CNC(=O)OC(C)(C)C)c(C(=O)c3ccccc3Cl)c2C1 ; "methanesulfonicacid" ; COC(=O)[C@@H]1Cc2sc(NC(=O)CN)c(C(=O)c3ccccc3Cl)c2C1 ; "pivalic acid" ; COC(=O)[C@@H]1Cc2sc3c(c2C1)C(c1ccccc1Cl)=NCC(=O)N3]

A solution of 50.00 kg (101.4 mol) compound 2b in 125 L toluene is heated to 60° C. in a first reactor. In a second reactor, a solution of 12.19 kg (126.8 mol) methanesulfonic acid in 100 L toluene and 50 L acetonitrile is heated to 60° C. At 60° C., the solution of compound 2b in toluene is added over a period of approximately 30 minutes to the solution in the second reactor. The first reactor is rinsed with 25 L toluene and further added. The resulting mixture is stirred for 1 hour at 60° C. The mixture is cooled to 55° C. before a solution of 15.00 kg (256.6 mol) sodium chloride and 21.33 kg (313.1 mol) aqueous ammonia solution (25%) in 125 L water is added. Additional 25 L of water are added, and the mixture is cooled to 45° C. The phases are separated, and the aqueous phase is discarded. 200 L of the organic solvent are distilled off under reduced pressure. 250 L of toluene are added, and again 200 L of the organic solvent are distilled off under reduced pressure. 150 L of 2-methylbutan-2-ol (tert-amyl alcohol) and 15.54 kg of (152.2 mol) pivalic acid are added. The reaction mixture is heated to 85° C. and stirred for 15 hours at this temperature. The mixture is cooled to 55° C. before a solution of 15.00 kg (256.6 mol) sodium chloride and 21.33 kg (313.1 mol) aqueous ammonia solution (25%) in 125 L of water is added. The mixture is cooled to 45° C., and 25 L of water are added. The phases are separated, and the aqueous phase is discarded. 200 L of the organic solvent are distilled off under reduced pressure. 250 L of acetonitrile are added before 230 L of the organic solvent are distilled off under reduced pressure. 150 L of acetonitrile are added, the mixture is cooled to 35° C., and the mixture is seeded with 50 g of compound 4a. Within a period of approximately 2 hours, the resulting suspension is cooled to 20° C. and stirred for 15 hours at this temperature. The mixture is then cooled to 0° C. within a period of approximately 2 hours and stirred for a minimum of 3 hours at this temperature. The product is collected via centrifugation, washed with 100 L of acetonitrile, and dried in a vacuum oven at 50° C.

Yield: 38.65 kg (72% of theory) of compound 4a as a solid.

¹H-NMR (400 MHz, DMSO-d₆) δ (ppm)=11.38 (s, 1H), 7.40-7.52 (m, 4H), 4.25 (s, 2H), 3.56 (s, 3H), 3.46-3.54 (m, 1H), 2.98-3.12 (m, 2H), 2.02-2.10 (m, 2H).

MS (ESI⁺/APCI⁺): 413 (M+H)⁺

What is claimed is:

1. A process for manufacturing alkyl (4S)-13-(2-chlorophenyl)-10-oxo-7-thia-9,12-diazatricyclo[6.5.0.0^{2,6}]trideca-1(8),2(6),12-triene-4-carboxylate (4) 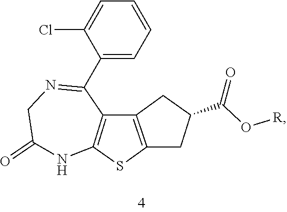
wherein R is C₁₋₃-alkyl,
comprising the following reaction steps:
(a) reacting compound 1, wherein R is C₁₋₃-alkyl, and N-(tert-butyloxycarbonyl)-glycine in the presence of a suitable amide coupling reagent in a suitable solvent to obtain compound 2, wherein R is C₁₋₃-alkyl and Boc is tert-butyloxycarbonyl; 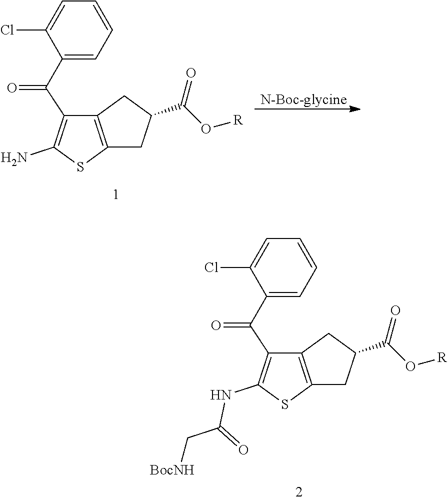
(b) optionally isolating compound 2;
(c) reacting compound 2, wherein R is C₁₋₃-alkyl and Boc is tert-butyloxycarbonyl, with a suitable acid in a suitable solvent to obtain compound 3, wherein R is C₁₋₃-alkyl; 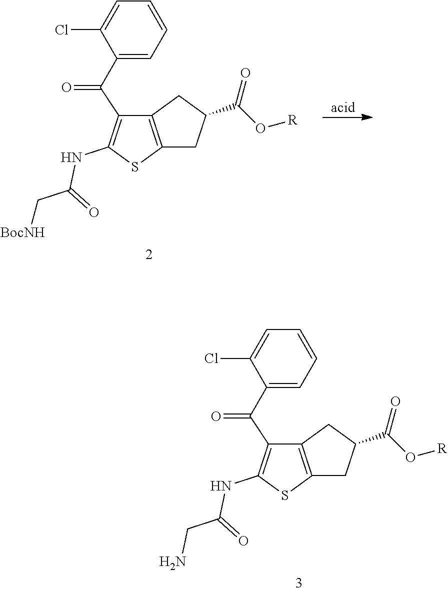
(d) optionally isolating compound 3;
(e) reacting compound 3, wherein R is C₁₋₃-alkyl, with a suitable acid in a suitable solvent to obtain compound 4, wherein R is C₁₋₃-alkyl; 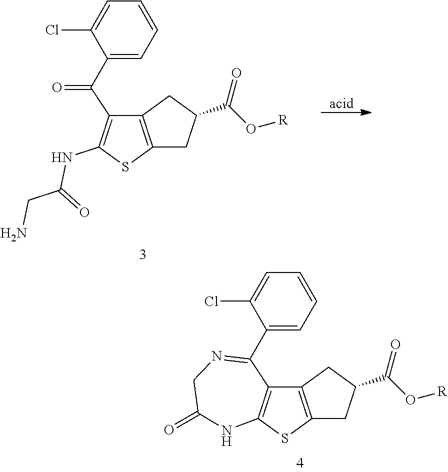
and
(f) isolating compound 4.

2. The process according to claim 1, characterized in that isolation step (b) is carried out prior to reacting compound 2 in step (c).

3. The process according to claim 1, characterized in that isolation step (d) is carried out prior to reacting compound 3 in step (e).

4. The process according to claim 1, characterized in that in step (a), 1,1′-carbonyldiimidazole, propanephosphonic acid anhydride, or 1-ethyl-3-(3-dimethylaminopropyl) carbodiimide is used as the amide coupling reagent.

5. The process according to claim 1, characterized in that in step (a), toluene, isopropyl acetate, N,N-dimethylformamide, or a mixture thereof is used as the solvent at a temperature between 50° C. and 110° C.

6. The process according to claim 1, characterized in that in step (b), compound 2 is precipitated via addition of an antisolvent and then isolated via filtration or centrifugation.

7. The process according to claim 1, characterized in that in step (c), methanesulfonic acid, 4-toluenesulfonic acid, or trifluoromethanesulfonic acid is used as the acid.

8. The process according to any claim 1, characterized in that in step (c), toluene, acetonitrile, methanol xylene, or a mixture thereof is used as the solvent.

9. The process according to claim 1, characterized in that in step (e), pivalic acid, benzoic acid, or 4-methylbenzoic acid is used as the acid.

10. The process according claim 1, characterized in that in step (e), 2-methylbutan-2-ol, methanol, acetonitrile, toluene, or a mixture thereof is used as the solvent at a temperature between 60° C. and 110° C.

11. The process according to claim 1, characterized in that in step (f), compound 4 is isolated via aqueous work up and subsequent recrystallization in a suitable solvent.

12. The process according to claim 1, further comprising the following steps to be carried out prior to step (a):
(g) reacting 3-(2-chlorophenyl)-3-oxo-propionitrile and (R)-alkyl-3-oxocyclopentan-1-carboxylate, wherein R is C₁₋₃-alkyl, in the presence of sulfur and a suitable base in a suitable solvent to obtain compound 1, wherein R is C₁₋₃-alkyl; 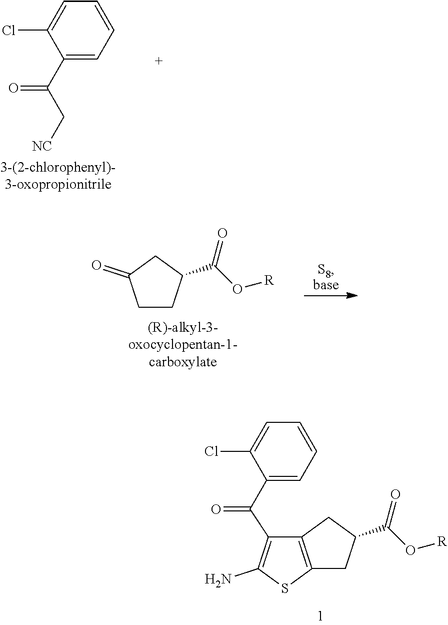
and
(h) optionally isolating compound 1.

13. The process according to claim 12, characterized in that isolation step (h) is carried out prior to reacting compound 1 in step (a).

14. The process according to claim 12, characterized in that in step (g) N,N-dimethylformamide, methanol, 1,3-dimethyl-2-imidazolidinone, or a mixture thereof is used as the solvent and morpholine, diisopropylethylamine, or N-methyl morpholine is used as the base.

15. The process according to claim 1, wherein R is methyl.

16. A compound of formula 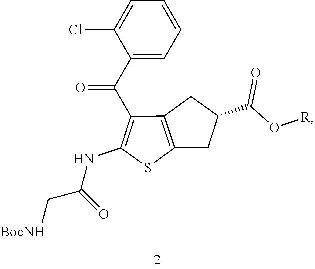
wherein R is C₁₋₃-alkyl, and
Boc is tert-butyloxycarbonyl.

17. The compound of claim 16, wherein R is methyl.
